(19) United States
(12) Patent Application Publication
(10) Pub. No.: US 2013/0123938 A1
(43) Pub. Date: May 16, 2013

(54) STITCHABLE TISSUE TRANSPLANT CONSTRUCT FOR THE RECONSTRUCTION OF A HUMAN OR ANIMAL ORGAN

(71) Applicant: Gouya Ram-Liebig, Dresden (DE)

(72) Inventor: Gouya Ram-Liebig, Dresden (DE)

(73) Assignee: UroTiss GmbH, Dresden (DE)

(21) Appl. No.: 13/575,914

(22) PCT Filed: Jan. 25, 2011
(86) PCT No.: PCT/DE2011/075010

§ 371 Date: Oct. 19, 2012

(30) Foreign Application Priority Data
Jan. 27, 2010 (DE) 10 2010 001 271.8

(51) Int. Cl. A61F 2/02

(52) U.S. Cl. CPC A61F 2/02
USPC 623/23.72

(57) ABSTRACT

The invention relates to a tissue transplant construct for the reconstruction of a human or animal organ. The tissue transplant construct (5) comprises
(a) a membrane composite (1) comprising at least a first biocompatible, collagen-containing membrane (2) and a second biocompatible, collagen-containing membrane (3), wherein the first membrane (2) and the second membrane (3) are adjacent to each other at their flat sides, and wherein the first membrane (2) is of equine or bovine origin and the second membrane (3) is of animal or human origin and the second membrane (3) is of another origin than the first membrane (2); and
(b) one or more layers (4) of mucosa tissue cells on one or both outer flat sides of the membrane composite (1).

The invention relates to a stitchable tissue transplant construct for the reconstruction of a human or animal organ, a method for the preparation of such a tissue transplant construct as well as a use of the tissue transplant construct. In particular, the invention relates to a tissue transplant construct for the reconstruction of a urinary organ, in particular the urinary bladder, ureter or urethra, esophagus, ophthalmic surface, or oral tissue defects.

One basic technical problem that must be prevented in the implantation of tissue transplant constructs based on a cellular tissue is the detachment of the tissue transplant construct due to the normal peristalsis of the organs, for example the urinary bladder, the ureter, or the esophagus. A further problem with the reconstruction of the ureter and the urethra is that the urinary flow requires an appropriate and firm fixation of the transplant to the wound bed. This applies also to the reconstructions in oral the region or in the region of the esophagus with respect to the intake of food and beverages. Such an appropriate and firm fixation of the transplant is necessary in the reconstruction of the ophthalmic surface due to the optokinesis.

Such an appropriate and firm fixation of the transplant can only be accomplished with a suitable suturing technique that ensures that with the transplant an adequate functional outcome is achieved, and that further prevents the risks of bleeding and infection. For the fixation of transplants the needle used has to be passed through the construct and the adjacent tissue to make a suture by means of the thread passed by the needle. The thread must be sufficiently tightened in order to achieve an appropriate coaptation of the adjacent tissue to the edge of the transplant. In case of an enlarged wound edema the tensile stress applied to the thread has to be increased. Moreover, for the reduction of the wound contraction there are required many times bigger sized needles and larger and deeper stitches.

Due to the previous facts a stability of the transplant is required which is so high that larger sized needles and larger and deeper stitches can actually be carried out without endangering the function or the existence of the transplant. Once a sufficient number of threads have been introduced between the transplant and the adjacent tissue there are typically tied knots with a sufficient stress to allow the approximation of the edges and thus prevent a dehiscence of the wound edges and cicatrization.

Due to the high tensile strength of the transplant that is required for the above-mentioned reasons the tissue transplant constructs must have a high mechanical stability. Up to now, different materials have been used that served as a framework for the tissue transplant constructs based on a cellular tissue. However, all these materials lack the necessary mechanical stability and stitchability. In most cases, only knots without tensile adaptation may be employed.

There are some membranes, such as small intestine submucosa, which have been reported to have a high mechanical strength. However, in the lyophilized form the three-dimensional collagen-structure of such membranes changes. That's why culturing of cells on long segments of said membranes to form multilayers is not possible (Wei R et al., Grafts of Porcine Small Intestinal Submucosa with Cultured Autologous Oral Mucosal Epithelial Cells for Esophageal Repair in a Canine Model., . . . experimental biology and medicine. 2009:234. 453-461: Lindberg K et al., Porcine small intestinal submucosa (SIS): a bioscaffold supporting in vitro primary human epidermal cell differentiation and synthesis of basement membrane proteins. Burns. 2003:254-266).

Further, there are known tissue transplant constructs comprising autologous oral mucosa cells cultured on an equine (TissuFoil) or bovine (Matriderm) collagenifibronectin matrix. The matrices are completely absorbed subsequent to tissue regeneration and correspond to complex surfaces. TissuFoil and Matriderm are certified medical products of Baxter and Suwelack and are approved for the use in humans. They allow a very good cell adhesion and proliferation on its surfaces.

EP 1 002 031 describes a stitchable membrane that is intended to prevent the adhesion of the membrane to prostheses or other compensatory parts. The membrane comprises a first layer of a collagen non-woven fabric and a spongy layer of collagen. The collagen of the two layers is derived from a common source. For example, the collagen used for both layers may be derived from cows, pigs, poultry, fish, rabbits, sheep, urine, and humans.

From US 2007/0161109 A 1 there is known an acellular membrane that is intended to promote accrementition. The membrane comprises a first layer and a second layer each having collagen fibers. The collagen fibers are derived from a native source not described in detail. Furthermore, a multilayered membrane of collagen from one and the same source is disclosed in U.S. Pat. No. 7,393,437.

However, with the action of tensile forces the mechanical stability of known matrices is insufficient for an effective fixation of tissue transplant constructs. There is a need for a tissue transplant construct with a higher tensile strength and improved stitchability. It is further desired to provide a tissue transplant construct that has a high biocompatibility and satisfies the medical law-related demands on its use in human.

The object of the invention is to eliminate the disadvantages according to the state of the art. In particular, there is provided a tissue transplant construct that has improved mechanical properties and at the same time is suitable for cultivation with cells. Furthermore, there are provided a method for the preparation of such a tissue transplant construct as well as uses of said tissue transplant construct.

This object is solved by the features of claims 1, 8, and 11. Practical embodiments of the invention result from the features of claims 2 to 7, 9 and 10 as well as 12 to 15.

According to the invention there is provided a tissue transplant construct for the reconstruction of a human or animal organ comprising
(a) a membrane composite comprising at least a first biocompatible, collagen-membrane and at least a second biocompatible, collagen-containing membrane, wherein the first membrane and the second membrane are adjacent to each other at their flat sides, and wherein the first membrane is of equine or bovine origin and the second membrane is of animal or human origin and the second membrane is of another origin than the first membrane; and
(b) one or more layers of mucosa tissue cells on one or both outer flat sides of the membrane composite.

The membrane composite can comprise one or more first membranes. In addition, the membrane composite can comprise one or more second membranes. In a preferred embodiment the membrane composite comprises two first membranes and one second membrane that is arranged between the two first membranes. In this way, a three-layered membrane composite is obtained which has a sandwich-like construction. Here, the upper side of the second membrane is covered with a first membrane with its lower side and the upper side of the second membrane facing each other. The lower side of the second membrane is covered with the other first membrane with its upper side and the lower side of the second membrane facing each other.

It is essential that the first membrane and the second membrane are different, i.e. of heterologous origin. Here, the term “origin” means that the first membrane and the second membrane are not derived from the same taxonomic species. A membrane of equine origin is derived from horse. A membrane of bovine origin is derived from cattle. A membrane of porcine origin is derived from pig.

The tissue transplant construct according to the invention offers improved mechanical properties, in particular an outstanding stitchability and mechanical stability, due to the use of a membrane composite to which on the one hand the good mechanical properties are attributable and which on the other hand has surfaces allowing a good adhesion of cells and their rapid proliferation to form dense layers on the outer surfaces of the membrane composite. In particular, the first membranes allow a good cell growth in vitro. For example, a good cell growth is present if one or more layers of cells grow on the outer surface of the first membrane, wherein the membrane surface should be >5 cm² the vitality of the cells at least 90% for a period of more than 48 hr.

The good mechanical properties are attributable to the properties of the second membrane, while the good properties with respect to the cell adhesion and proliferation are attributable to the properties of the first membrane. The second membrane should not be waterproof. When the second membrane is water-permeable this promotes the flow of wound exudations through the membrane composite, so that the mucosa tissue cells of the tissue transplant construct as well as the cells adjacent to or penetrated in the tissue transplant construct can be reached by the wound exudations. Additionally, the liquid patency of the second membrane prevents the formation of edema and the associated separation of the implant from the wound bed.

The tissue transplant construct according to the invention has a high biocompatibility and complies with the statutory requirements for medical products.

Preferably, the first membrane is of equine or bovine origin. In addition to collagen, in particular collagen fibers, the first membrane can contain further constituents such as for example fibronectin. Suitable first membranes are the equine collagen-containing membranes marketed under the trade name “TissuFoil” (manufacturer: Baxter Deutschland GmbH, DE) as well as collagen fibronectin membranes marketed under the trade name “Matriderm” (manufacturer: Dr. Suwelack Skin & Health Care AG, DE).

A preferred second membrane is a membrane that is derived from a warm-blooded animal or a human. More preferably, the second membrane is a membrane of porcine origin. A particularly suitable second membrane is a porcine small intestine submucosa, in particular a lyophilized porcine small intestine submucosa. Besides collagen, in particular collagen fibers, the second membrane can contain further constituents like glycoproteins, proteoglycans, and glycosaminoglycans.

The first membrane may be one or multi-ply. Moreover, the second membrane may be one or multi-ply. In particular, the second membrane may be one to four-ply. Herein, a membrane is in particular understood as a flat porous structure.

The membrane composite is composed of membranes of biological and not synthetic origin. As a result, the membrane composite of the tissue transplant construct according to the invention is unlike vascular grafts completely degradable preventing a calcification or rejection of the tissue transplant construct in the period after implantation. The presence of vital cells on the membrane allows the generation of new tissue by endogenous cells. This eliminates the need of a permanent durability of the prosthesis and the support material, respectively.

The inventor of the present invention has surprisingly found that a membrane composite of the two collagen-containing first and second membranes may be prepared by compressing the first and second membranes and/or by cross-linking, in particular by photocrosslinking, the first and second membranes. A membrane composite thus obtained has a sufficient mechanical stability, so that it can be used for the production of a tissue transplant construct that in particular may be employed as a replacement for epithelial tissue of an animal and/or human organ. Thus, the membrane composite according to the invention and a tissue transplant construct prepared by using the membrane composite according to the invention are particularly suitable for the reconstruction of epithelial tissue. In particular, the tissue transplant construct according to the invention is suitable for the reconstruction of a urinary organ, in particular the urinary bladder, the ureter, or the urethra as well as the esophagus, the ophthalmic surface, or oral tissue defects.

Preferably, the mucosa tissue cells provided in accordance to the invention are autologous mucosa tissue cells, more preferably autologous oral mucosa tissue cells. Due to the use of autologous mucosa tissue cells the tissue transplant constructs according to the invention are particularly suitable for repair and/or replacement of epithelial tissue.

Particularly suitable autologous oral mucosa tissue cells are oral mucosa tissue cells. Oral mucosa tissue cells have a strong proliferation potential and are available via relatively non-invasive biopsies making the oral mucosa epithelial tissue an attractive source of cells for autologous therapies.

Details of the autologous oral mucosa tissue cells can be found in the following section “autologous oral mucosa tissue cells.”

According to the invention there is further provided a method for the preparation of a tissue transplant construct comprising the steps of:
(a) preparing a membrane composite comprising at least a first biocompatible, collagen-containing membrane and a second biocompatible, collagen-containing membrane, wherein the first membrane and the second membrane are adjacent at their flat sides and wherein the first membrane is of equine or bovine origin and the second membrane is of animal or human origin, and the second membrane is of another origin than the first membrane; and
(b) forming one or more layers of mucosa tissue cells on one or both outer flat sides of the membrane composite.

Here, step (a) of this method preferably comprises the following individual steps of:
(a1) providing two first membranes and converting the first membranes into a swollen state;
(a2) providing a second membrane and lyophilizing the second membrane;
(a3) arranging the second membrane between the two first membranes; and
(a4) preparing a composite of the first membranes and the second membrane.

Preferably, the membrane composite is prepared by compressing and/or cross-linking the first and second membranes. Compressing is preferably carried out under a pressure of 5 to 5000 kN/cm², more preferred 10 to 1000 kN/cm² and still more preferred at 50 to 150 kN/cm² and most preferred at 100 kN/cm². At pressures of less than 5 kN/cm² possibly no sufficiently firm composite of the membranes can be achieved, whereas at pressures above 5000 kN/cm² the membranes, in particular their framework and pore structure can be damaged.

Preferably, compressing is performed at a temperature that slightly increased over the ambient temperature, preferably at 25 to 50° C., more preferred at 35 to 40° C. The slightly increased temperature promotes the mobility of the collagen fibers without changing their three-dimensional structure. Preferably, compressing is performed over a period of 10 min. to 2 hr, more preferred 0.5 to 1.5 hr, and particularly preferred 3 to 13 minutes.

Alternatively or in addition to compressing the membrane composite may be subjected to a photochemical treatment to achieve cross-linking of the collagen fibers of the first and second membranes. The method to photochemically crosslink collagen fibers is in particular known from the ophthalmology for the treatment of keratoconus. The method of cornea collagen cross-linking there consists of the photo-polymerization of stroma fibers by the combined effect of a photosensitizing substance (riboflavin or vitamin B2) and ultraviolet A rays (UVA). The photo-polymerization increases the stiffness of corneal collagen and its resistance to keratectasia (s., for example: Corneal collagen cross-linking with riboflavin and ultraviolet-A light for keratoconus: Results in Indian eyes. Agrawal V. Indian Journal of Ophthalmology, 2008:57(2).111-114).

Preferably, photo-chemical cross-linking is performed with visible light. The light has preferably a wavelength of from 380 to 600 nm, more preferred 425 to 525 nm, particularly preferred 475 nm. Cross-linking is preferably carried out over a period of 10 min, to 2 hr, particularly preferred 0.5 to 1.5 hr.

### Autologous Oral Mucosa Tissue Cells

### a) Use of Autologous Oral Mucosa Cells in the Urological Reconstruction

Urethral and ureteral strictures are constrictions of the organ caused by inflammation, cicatritial tissue, permanent catheter, instrumentation, external trauma, operations. In this case, cicatritial tissue replaces the normal urethral or ureteral epithelial tissue. Open urethroplasty and ureteroplasty are considered as the gold standard treatment of urethral and ureteral stricture. Oral mucosa transplants are recognized as the most promising replacement in urologic organs. However, donor site morbidity at oral sites is a main concern.

Since the first report of an in-vitro culture of transitional cell epithelium by Bunge in 1955 tissue technology in urological reconstruction has covered a long way. Of course, in order to obtain a successful substitutive urethroplasty a tissue technology-based product should have a matrix that is biocompatible and robust and stitchable under traction, and at the same time allows the optimum delivery of cells to the place of the urethroplasty and also the adequate fixation of the transplant at the implantation site and wound stabilization. While cultured oral mucosa cells represent optimum cell candidates for tissue technology-based urologic organs in clinical and experimental arrangements several materials, both organic and synthetic, are used to provide an urethra and ureter replacement; these materials contain acellular bladder matrices, acellular porcine small intestine submucosa (SIS), tissues of Dexon, collagen matrices, and polytetrafluoroethylene (GORE-TEX) (S.: Roinagnoll G et al., Onestep treatment of proximal hypospadias by the autologous graft of cultured urethral epithelium. Journal of Urology. 1993:150(4).1204-1207 . . . El-Kassaby A W et al., Urethral stricture repair with an off-the-shelf collagen matrix. Journal of urology. 2003:169(1).170-173 . . . Badylak S F et al. The extracellular matrix as a biologic scaffold material. Biomaterials. 2007:28.3587-3593). As a rule, these materials had limited success due to their mechanical, structural, or biocompatibility problems. Recently it has been shown that SIS not colonized with cells are substantially more promising due to their mechanical breaking strength, however, good results were only achieved in patients with defects at short urethral segments (s.: Bhargava et al.,. Tissue-Engineered Buccal Mucosa Urethroplasty-Clinical Outcomes. European Urology. 2008: 53(6).1263-1271). In patients having longer or anuria the reepithelialization of protein scaffolds not colonized with cells was never completely successful (s.: Palminteri E et al. Small intestinal submucosa (SIS) graft urethroplast: shortterm results. European Urology. 2007:51(6).1695-1701 Fiala R et al.,. Porcine small intestinal suhmucosa graft for repair of anterior urethral strictures. European Urology. 2007:51(6).1702-1708), Bhargava et al., reported the clinical course of a technique for tissue technology-based autologous buccal mucosa on a de-epithelialized, sterilized donor skin matrix in the substitutive urethroplasty. The protein scaffold used in this study (de-epithelialized dermis) was obtained from shielded organ donors via the National Blood Service Skin Bank. However, the protein scaffold requires cadaver material, which is scarce. Moreover, the study results of the urethroplasty in men were not the best. In this publication an early inflammatory reaction against the protein scaffold as another reason for transplant contraction and fibrosis was discussed. Actually, a protein scaffold-like de-epithelialized dermis in vivo is not degraded in a short time (1 to 2 weeks) and thus, as a result a strong inflammatory reaction can be induced in the body.

### b) Use of Autologous Oral Mucosa Cells for the Ophthalmic Surface Reconstruction

A severe disease of the ophthalmic surface caused by conditions such as the Stevens-Johnson syndrome and ocular cicatricial pemphigoid is a potentially destructive condition with significant visual morbidity. In such cases, the corneal epithelial stem cells in the limbus are destroyed resulting in the invasion of the ectocornea by surrounding conjunctiva, neovascularization, chronic inflammation, ingrowth of fibrous tissue, and stroma cicatrization. With these patients the conventional transplantation of the cornea is associated with awful results. Alternative methods such as the transplantation of cultured corneal epithelial stem cells have been demonstrated. In this way, patients having a unilateral damage of the cornea received transplants of cultured corneal epithelial stem cells obtained from the healthy contralateral eye. However, health of the eye is a main concern. In patients having a bilateral damage of the eye transplantation of cultured corneal epithelial stem cells of cadaver donors or a living donor eye is required. Despite some success, immunologic rejection and microbial infection as a result of an immunosuppressive therapy following allogenic transplantation continues to pose a challenge. In context of regenerative medicine, transplantation of cultured mucosa epithelial stem cell layers generated from autologous cellular sources represents a developable alternative in cases of bilateral damage of the eye, which invalidates the use of autologous corneal epithelial stem cells. Oral mucosa cells have attracted attention as a cellular source and in animal and preliminary human pilot studies positive results were obtained. This method reduces the risk for transplant rejection and the necessity of long-term steroids or immunosuppression. (s.: Midterm results on ocular surface reconstruction using cultivated autologous oral mucosa epithelial transplantation. Inatomi T, Nakamura T, Koizumi N, . . . American Journal of ophthalmology. 2006:141(2). 267-276. The use of autologous serum in the development of corneal and oral epithelial equivalents in patients with Steven Johnson Syndrome. Nakamura T, Ang L, Rigby H, Sekiyama E, . . . Investigative ophthalmology and visual science. 2006:47(3). 909-914). The presently preferred method for culturing corneal or oral epithelial cells requires the use of mechanically instable materials, often an amniotic membrane (s.: Inatomi et al., Nakamura et al., supra). The use of an amniotic membrane also requires the allogenic placenta of women who had a caesarean section, wherein here is a lack of material. This is also a problem in other proposed oral mucosa cell constructs, such as EVPOME that also requires cadaver material (s.: 3 Clinical and Histopathological Analysis of Healing Process of Intraoral Reconstruction with ex vivo Produced Oral Mucosa Equivalent. HOTTA T, YOKOO 5, TERASHI H. Kobe Journal of medical science. 2007: 53(1).1-14).

### c) Use of Autologous Oral Mucosa Cells the Reconstruction of Esophagus

The acellular matrices have been used for the oesophagoplasty in animal models. However, this did not result in a complete epitheliogenesis. Thus, for better reconstruction a cellular component is required. In animal models the use of oral mucosa epithelial cells on acellular small intestine submucosa showed promising results in the reconstruction of short esophagus defects of about 5 cm. However, due to the lyophilized form of the small intestine submucosa a multi-ply culture of cells on longer segments of the membrane is not always possible (s.: Grafts of Porcine Small Intestinal Submucosa with Cultured Autologous Oral Mucosa Epithelial Cells for Esophageal Repair in a Canine Model. Wei R. Tan D. Tan M, Luo J, Deng L, . . . Experimental biology and medicine. 2009:234. 453-461). Also other reported membranes for the oesophagoplasty that consist of collagen are mechanically too instable for use in humans (s.: Esophagus Tissue engineering: in vitro generation of esophageal epithelial cell sheets and viability on scaffold. Saxenu A Ainoedhofer H, Höllwarth M. Journal of pediatric surgery. 2009:44. 896-901). Further synthetic materials for oesophagoplasty such as poly(l-lactic acid) (PLLA), poly(lactic-co-glycol)acid (75: 25) (PLGA75), poly(lactic-co-glycol)acid (50:50) (PLGA50), and polycaprolactone/poly(l-lactic acid) (50:50) (PCL/PLLA) have proven to be unsuitable for tissue technology with respect to the esophagus (s.: Esophageal epithelial cell interaction with synthetic and natural scaffolds for tissue engineering. Beckstead B^{a}, Pan 5, Bhrany A. Bratt-Leal A, . . . Biomaterials. 2005: 26(31).6217-622),

### d) Use of Autologous Oral Mucosa Cells for Dermal Reconstruction (Burns)

Oral keratinocytes have several unique features that may offer advantages over epidermal (skin) keratinocytes. Oral keratinocytes have a higher rate of proliferation and a lower rate of terminal differentiation than epidermal keratinocytes. For this reason, relatively small donor sites may provide sufficient cell mass for covering much bigger wounds by means of ex vivo expansion. Moreover, oral keratinocytes secrete pro-angiogenic factors such as VEGF and IL8 which promote their rapid integration at the transplantation sites. Thus, oral mucosa cells have proven to be suitable candidates for dermal reconstruction (s.: Development of a tissue-engineered human oral mucosa equivalent based on an acellular allogeneic dermal matrix: A preliminary report of clinical application to burn wounds. Takuya L, Takami V. Yamaguchi R, Shimazaki 5, . . . Scandinavian Journal of Plastic and Reconstructive Surgery and Hand Surgery. 2005: 39(3).138-146). As mentioned above, the disadvantage of said construct is the use of human cadaver materials.

Hereinafter, the invention is explained in more detail with the help of examples not intended to limit the invention with respect to the drawings. Here

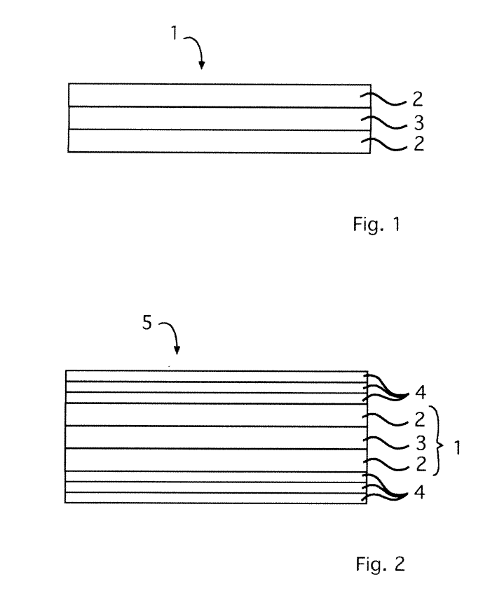

FIG. 1 shows a schematic representation of a first embodiment of a membrane composite according to the invention in cross-section;

FIG. 2 shows a schematic representation of a first embodiment of a tissue transplant construct according to the invention;

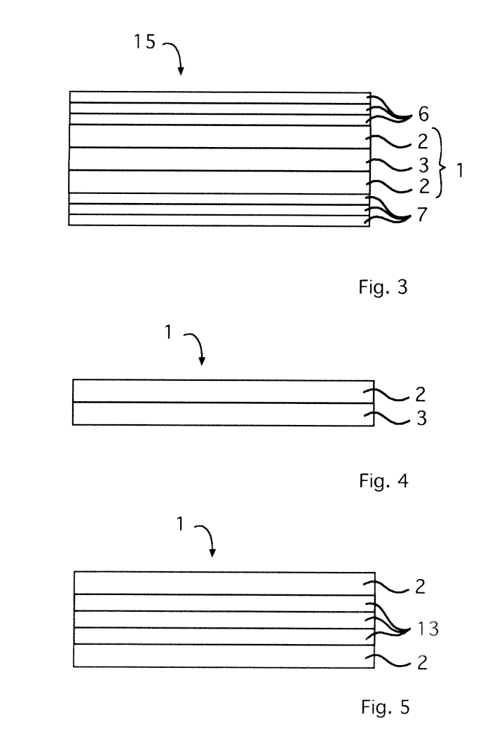

FIG. 3 shows a schematic representation of a second embodiment of a tissue transplant construct according to the invention;

FIG. 4 shows a schematic representation of a second embodiment of a membrane composite according to the invention in cross-section; and

FIG. 5 shows a schematic representation of a third embodiment of a membrane composite according to the invention in cross-section.

### EXAMPLES

### Example 1

### Preparation of a Membrane Composite

A membrane composite having two first membranes and one second membrane was prepared wherein the second membrane was arranged between the two first membranes.

As the first membrane two biodegradable equine collagen membranes (trade name “TissuFoil” by Baxter Deutschland GmbH, DE) or two bovine collagen/fibronectin membranes (trade name “Matriderm” by Dr. Suwelack Skin & Health Care AG, DE) have been used. These membranes are certified medical products and are thus allowed for use in patients.

As the second membrane a lyophilized porcine small intestine submucosa has been used which in particular contained collagen, glycoproteins, proteoglycans, and glycosaminoglycans.

The two first membranes were placed in phosphate buffered saline for 24 hr to convert them into a swollen and porous form. Subsequently, the second membrane is placed between the two first membranes. The thus obtained three-ply structure was compressed at a temperature of 35 to 40° C. under a pressure of 100 kN'cm² to obtain the membrane composite (duration of compression: 5 minutes). Subsequently, the membrane composite was soaked with 5% vitamin (riboflavin) solution for one hour. Afterwards, the membrane composite was treated with visible light with a wavelength of 475 nm for one hour to increase the stiffness of the membrane composite and reduce its contraction once it has been implanted.

Membrane composites of the following structure have been obtained:
1.1: equine membrane porcine small intestine submucosa equine membrane
1.2: bovine membrane/porcine small intestine submucosa/bovine membrane

The structure of membrane composite 1.1 and the structure of membrane composite 1.2 are schematically shown in cross-section in FIG. 1. The membrane composite 1 represented there has two first membranes 2 and one second membrane 3. The second membrane 3 is arranged between the two first membranes 2 in a sandwich-like construction.

### Example 2

### Preparation of a Tissue Transplant Construct according to the Invention for Use as Mucosa Transplant

Membrane composites prepared in accordance to example 1 (membrane composite 1.1 or membrane composite 1.2) were sown with mucosa keratinocytes to prepare tissue transplant constructs according to the invention to be used as mucosa transplants. For that, a biopsy specimen of 2 to 4 mm in diameter was taken from the buccal mucosa of 40 patients. Additionally, 30 ml of autogenic serum were extracted from a venous whole blood sample of these patients. The primary cultures were incubated in Dulbecco's Modified Eagle Medium and nutrient factor F 12 (Gibco Eggenstein, Germany) that contained the conventional additives and autogenic serum (s., Lauer G, Schimming R, Klinische Anwendung von im Tissue Engineering gewonnenen autologen Mundschleimhauttransplantaten. Mund Kiefer Gesichtschirurgie. 2002. 6:379-393) for three weeks in a known matter (s., Lauer G, Schimming R, Klinische Anwendung von im Tissue Engineering gewonnenen autologen Mundschleimhauttransplantaten. Mund Kiefer Gesichtschirurgie.2002. 6:379-393). Subsequently, to create mucosa transplants having several layers of oral mucosa cells, was sub-cultured on the membrane composite obtained in example 1.

The incubated mucosa tissue cells were added to both flat sides of the membrane composite and cultured there. After 48 hr cell distribution analysis with MTT dye showed a membrane covering of >90% on both sides of the membrane composite. Assays with respect to the viability of the cells with calcein/ethidium bromide fluorescent dyes showed a cell viability on the membrane of >90%. Moreover, >30% of the cells showed a positive reaction with bromodeoxyuridine (BrdU) and thus had proliferation capability.

Tissue transplant constructs of the following structure have been obtained:
2.1: one or more layers of mucosa tissue cells/equine membrane/porcine small intestine mucosa/equine membrane/one or more layers of mucosa tissue cells
2.2: one or more layers of mucosa tissue cells/bovine membrane/porcine small intestine submucosa/bovine membrane/one or more layers of mucosa tissue cells

In FIG. 2, the structure of a tissue transplant construct 5 prepared in accordance to example 2 is schematically shown in cross-section. The outer flat sides of the membrane composite shown in FIG. 1, so also with respect to FIG. 1 its upper side and lower side, are covered with several layers 4 formed of mucosa tissue cells.

### Example 3

In the same manner as in example 2 both flat sides of the membrane composites obtained in accordance to example 1 were sown with different cell cultures. Here, on one outer flat side of the respective membrane composite keratinocytes were cultured whereas on the other flat side of the membrane composite a mixture of oral tissue fibroblasts and mucosa tissue endothelial cells was cultured (source of the cells: oral mucosa tissue biopsy; mixing ratio between the oral fibroblasts and the endothelial cells 1:3.) Following the keratinocytes colonization of one flat side of the membrane composite this mixed population was attached to the other flat side of the membrane composite after 30 minutes. For the rest, the procedure for culturing cells corresponds to that of example 2.

Tissue transplant constructs of the following structure have been obtained:
2.1: one or more layers of keratinocytes/equine membrane porcine small intestine submucosa equine membrane/one or more layers of a mixture of oral fibroblasts and endothelial cells
2.2: one or more layers of keratinocytes/bovine membrane/porcine small intestine submucosa/bovine membrane/one or more layers of a mixture of oral fibroblasts and endothelial cells

In FIG. 3, the structure of a tissue transplant construct 15 prepared in accordance to example 3 is schematically shown in cross-section. The upper flat side 1 of the membrane composite) shown in FIG. 1 is covered with several layers 6 formed of keratinocytes. The lower flat side of the membrane composite 1 is covered with several layers 7 formed of a mixture of oral fibroblasts and endothelial cells.

### Comparative Example 1

For comparison the cell cultures described in example 2 and example 3 were applied to comparative membranes. As the comparative membranes there were used: i) porcine small intestine submucosa; ii) an equine membrane (trade name: “TissuFoil”) and iii) a bovine membrane (trade name: “Matriderm”). The comparative membranes correspond to the first or second membranes used in example 1, except that no composite membrane was prepared. The conditions for culturing the cells were the same as in example 2. It was found that the cell growth on an untreated equine membrane (TissuFoil) or bovine membrane (Matriderm) was similar to the culturing process on a membrane composite according to the invention described in example 2, whereas, when using small intestine submucosa as the membrane, a reduced membrane covering (approx. 50%) with a cell viability of about 70% and a proliferation capacity of <10% was observed.

The following comparative constructs have been obtained:

i) one or more layers of mucosa tissue cells/porcine small intestine submucosa one or more layers of mucosa tissue cells
ii) one or more layers of mucosa tissue cells/equine membrane/one or more layers of mucosa tissue cells
iii) one or more layers of mucosa tissue cells/bovine membrane/one or more layers of mucosa tissue cells

### Example 4 and Comparative Example 2

### Mechanical Properties of the Tissue Transplant Constructs According to the Invention

Stability and tensile strength of the tissue transplant constructs prepared in example 2 and comparative example 1 were examined 48 hours after cell seeding. It was found that the tissue transplant constructs according to the invention prepared in example 2 did not tear and at the same time showed outstanding stitchability, tensile strength, unthread strength, and knot application strength. The comparative constructs with small intestine submucosa prepared in accordance to comparative example 1 showed a similar mechanical stability. Comparative constructs with the equine membranes (TissuFoil) and bovine membranes (Matriderm) also prepared in accordance to comparative example 1 showed a reduced mechanical stability and slightly tare under tension, in the unthread treatment, or knot application.

### Examples 5 and 6

### Construction of Further Membrane Composites

The membrane composite 1 shown in FIG. 4 in contrast to the membrane composite shown in FIG. 1 has only one first membrane 2. With that, only one flat side of the second membrane is covered. The membrane composite 1 shown in FIG. 5 in contrast to the membrane composite shown in FIG. 1 has a multi-ply second membrane 13. Apart from this, structure and preparation of these membrane composites correspond to that of example 1.

### LIST OF REFERENCE MARKS

1 Membrane Composite
2 First Membrane
3 Second Membrane
4 Layers of Mucosa Tissue Cells
5 Tissue Transplant Construct
6 Layer of Keratinocytes
7 Layer of a Mixture of Oral Fibroblasts and Endothelial Cells
13 Mulit-ply Second Membrane
15 Tissue Transplant Construct

1. A tissue transplant construct the reconstruction of a human or animal organ comprising
(a) a photo-chemically cross-linked membrane composite (1) comprising at least a first biocompatible, collagen-containing membrane (2) and at least a second biocompatible, collagen-containing membrane (3), wherein the first membrane (2) and the second membrane (3) are adjacent to each other at their flat sides, and wherein the first membrane (2) is of equine or bovine origin and the second membrane (3) is of animal or human origin and the second membrane (3) is of another origin than the first membrane (2); and
(b) one or more layers of autologous oral mucosa tissue cells (4, 5, 6) on one or both outer flat sides of the membrane composite (1).

2. The tissue transplant construct according to claim 1, characterized in that the membrane composite (1) comprises two first membranes (2) and one second membrane (3), wherein the second membrane (3) is arranged between the two first membranes (2).

3. The tissue transplant construct according to claim 1, characterized in that the first membrane (2) is of equine or bovine origin and the second membrane (3) is of porcine origin.

4. The tissue transplant construct according to claim 1, characterized in that the first membrane (2) in addition to collagen also contains fibronectin.

5. The tissue transplant construct according to claim 1, characterized in that the first and second membranes (2, 3) of the membrane composite (1) are compressed and/or cross-linked with each other.

6. A membrane composite for a tissue transplant construct comprising at least one first biocompatible, collagen-containing membrane (2) and a second biocompatible, collagen-containing membrane (3), wherein the first membrane (2) and the second membrane (3) are adjacent to each other at their flat sides, and wherein the first membrane (2) is of equine or bovine origin and the second membrane (3) is of animal or human origin and the second membrane (3) is of another origin than the first membrane (2), wherein the first and the second membrane are photo-chemically cross-linked.

7. The membrane composite according to claim 6, characterized in that it comprises two first membranes (2) and one second membrane (3), wherein the second membrane (3) is arranged between the two first membranes (2).

8. The membrane composite according to claim 6, characterized in that the first and second membranes (2, 3) of the membrane composite (1) are compressed and/or cross-linked with each other.

9. A method for the preparation of a tissue transplant construct according to claim 1 comprising
(a) the preparation of a membrane composite (1) comprising at least one first biocompatible, collagen-containing membrane (2) and a second biocompatible, collagen-containing membrane (3), wherein the first membrane (2) and the second membrane (3) are adjacent to each other at their flat sides, and wherein the first membrane (2) is of equine or bovine origin and the second membrane (3) is of animal or human origin and the second membrane (3) is of another origin than the first membrane, to photo-chemically cross-link the first and second membranes; and
(b) the formation of one or more layers (4) of autologous oral mucosa tissue cells on one or both of the outer flat sides of the membrane composite (1).

10. The method according to claim 9, characterized in that step (a) comprises the steps of
(a1) providing two first membranes (2) and converting the first membranes 2 into a swollen state;
(a2) providing a second membrane (3) and lyophilizing the second membrane (3);
(a3) arranging the second membrane (3) between the two first membranes (2): and
(a4) preparing a composite (1) of the first membranes (2) and the second membrane (3).

11. The method according to claim 9, characterized in that the membrane composite (1) is prepared by compressing and/or cross-linking the first and the second membranes (2, 3).

12. The method according to claim 9, characterized in that autologous oral mucosa tissue cells are applied onto one or both of the outer flat sides of the membrane composite (1) and there are cultured to form one or more layers (4).

13. The tissue transplant construct according to claim 2, characterized in that the first membrane (2) is of equine or bovine origin and the second membrane (3) is of porcine origin.

14. The membrane composite according to claim 7, characterized in that the first and second membranes (2, 3) of the membrane composite (1) are compressed and/or cross-linked with each other.

15. The method according to claim 10, characterized in that the membrane composite (1) is prepared by compressing and/or cross-linking the first and the second membranes (2,3).

16. A method for the preparation of a tissue transplant construct according to claim 9, wherein the membrane composite (1) comprises two first membranes (2) and one second membrane (3), wherein the second membrane (3) is arranged between the two first membranes (2).

17. A method for the preparation of a tissue transplant construct according to claim 9, wherein the first membrane (2) is of equine or bovine origin and the second membrane (3) is of porcine origin.

18. A method for the preparation of a tissue transplant construct according to claim 9, wherein the first membrane (2) in addition to collagen also contains fibronectin.

19. A method for the preparation of a tissue transplant construct according to claim 9, wherein the first and second membranes (2, 3) of the membrane composite (1) are compressed and/or cross-linked with each other.
